(19) United States
(12) Patent Application Publication
(10) Pub. No.: US 2020/0008734 A1
(43) Pub. Date: Jan. 9, 2020

(54) METHOD AND SYSTEM FOR NAVIGATING A USER FOR CORRECTING A VESTIBULAR CONDITION

(71) Applicants: Rajneesh Bhandari, Jaipur (IN); Anita Bhandari, Jaipur (IN)

(72) Inventors: Rajneesh Bhandari, Jaipur (IN); Anita Bhandari, Jaipur (IN)

(21) Appl. No.: 16/197,561

(22) Filed: Nov. 21, 2018

(30) Foreign Application Priority Data
Jul. 5, 2018 (IN) 201811017105

(51) Int. Cl. A61B 5/00; G16H 50/50

(52) U.S. Cl. CPC A61B 5/4023; A61B 5/6813; A61B 5/72; G16H 20/30; A61B 5/743; G16H 50/50; A61B 5/744

(57) ABSTRACT

The invention provides a method and system for navigating a user based on a type of maneuver for correction of a vestibular condition. The method and system collects sensor data regarding orientation of head and body of a person for creating a three-dimensional model of the person. The method and system then generates a sequence of steps corresponding to the type of maneuver along with an instruction set and a time duration for performing each step of the sequence of steps, thus enabling the user to perform each step of the sequence of steps corresponding to the type of maneuver, on the person for correcting the vestibular condition.

### FIELD OF THE INVENTION

The invention generally relates to a method and system for correcting vestibular conditions and similar disorders. More specifically, the invention relates to a method and system for navigating a user based on a type of maneuver for correction of a vestibular condition and similar disorders.

### BACKGROUND OF THE INVENTION

One of the very common and prevalent causes of vertigo and other balance related disorders include the Benign Paroxysmal Positional Vertigo (BPPV). The usual symptoms of imbalance/spinning sensation usually occur when a person changes a position as some of the calcium carbonate crystals (otoconia) that are typically embedded in the gel in the utricle become displaced and migrate into one or more of the three fluid-filled semicircular canals. Another symptom accompanying the usual symptoms includes abnormal rhythmic eye movements called nystagmus.

Reoccurrence of the calcium carbonate crystals in the three fluid-filled semicircular canals even after performance of the existing types of maneuvers often owes its existence to the lack of precision and accuracy maintained in the performance of the steps associated with the types of maneuvers, by a user.

Furthermore, the performance of steps associated with the types of maneuvers includes extensive training of users to try and maintain precision and therefore requires extensive investments in creating a specialized trained skill set.

Also, existing techniques involve the use of mechanized chairs for performing the maneuver, which are bulky and expensive.

Therefore, in light of the above, there is a need for a method and system that provides a cost-effective and accurate system while navigating a user through a type of maneuver for appropriate correction of vestibular conditions.

### BRIEF DESCRIPTION OF THE FIGURES

The accompanying figures where like reference numerals refer to identical or functionally similar elements throughout the separate views and which together with the detailed description below are incorporated in and form part of the specification, serve to further illustrate various embodiments and to explain various principles and advantages all in accordance with the invention.

FIG. 1 illustrates a system for navigating a user based on a type of maneuver for correction of a vestibular condition in accordance with an embodiment of the invention.

FIG. 2 illustrates a flowchart depicting a method for navigating a user based on a type of maneuver for correction of a vestibular condition in accordance with an embodiment of the invention.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of embodiments of the invention.

### DETAILED DESCRIPTION OF THE INVENTION

Before describing in detail embodiments that are in accordance with the invention, it should be observed that the embodiments reside primarily in combinations of method steps and system components related to navigating a user in accordance with a type of maneuver for correcting the vestibular condition experienced by a person and providing a feedback for increasing the level of accuracy.

Accordingly, the system components and method steps have been represented where appropriate by conventional symbols in the drawings, showing only those specific details that are pertinent to understanding the embodiments of the invention so as not to obscure the disclosure with details that will be readily apparent to those of ordinary skill in the art having the benefit of the description herein.

In this document, relational terms such as first and second, top and bottom, and the like may be used solely to distinguish one entity or action from another entity or action without necessarily requiring or implying any actual such relationship or order between such entities or actions. The terms “comprises,” “comprising,” or any other variation thereof, are intended to cover a non-exclusive inclusion, such that a process, method, article or composition that comprises a list of elements does not include only those elements but may include other elements not expressly listed or inherent to such process, method, article or composition. An element proceeded by “comprises . . . a” does not, without more constraints, preclude the existence of additional identical elements in the process, method, article or composition that comprises the element.

Various embodiments of the invention provide a method and system for navigating a user based on a predetermined type of maneuver for correction of a vestibular condition. A sensor device, communicatively coupled to a memory and processor, is configured to collect sensor data regarding a head orientation and a body orientation of a person experiencing a vestibular condition. In accordance with an embodiment, in addition to the sensor device, the method and system includes another sensor device to monitor eye movements, specifically eye nystagmus and torsional eye movements of the person in real-time. Based on the collected sensor data, one or more processors are configured to create a three-dimensional model of the person in accordance with the head orientation, the body orientation and the eye movements of the person. Further, a sequence of steps is generated in accordance with the predetermined type of maneuver, wherein each step of the sequence of steps is associated with an instruction set and a time duration for performing the step. The time duration for performing each step is computed by a time computation module. Once the sequence of steps is generated, the one or more processors enable the user to perform each step of the sequence of steps by displaying an animation corresponding to each step to be performed by the user, on a display device. An animation corresponding to a step is further overlaid on the three-dimensional model of the person. The method and system further includes a feedback module, communicatively coupled to the memory and the processor for providing a real-time feedback based on change in real-time eye nystagmus and torsional eye movements during the performance of the sequence of steps corresponding to the maneuver based on a change in real-time eye nystagmus and torsional eye movements at the end of performance of each step, thereby ensuring high accuracy levels in the performance of the type of maneuver.

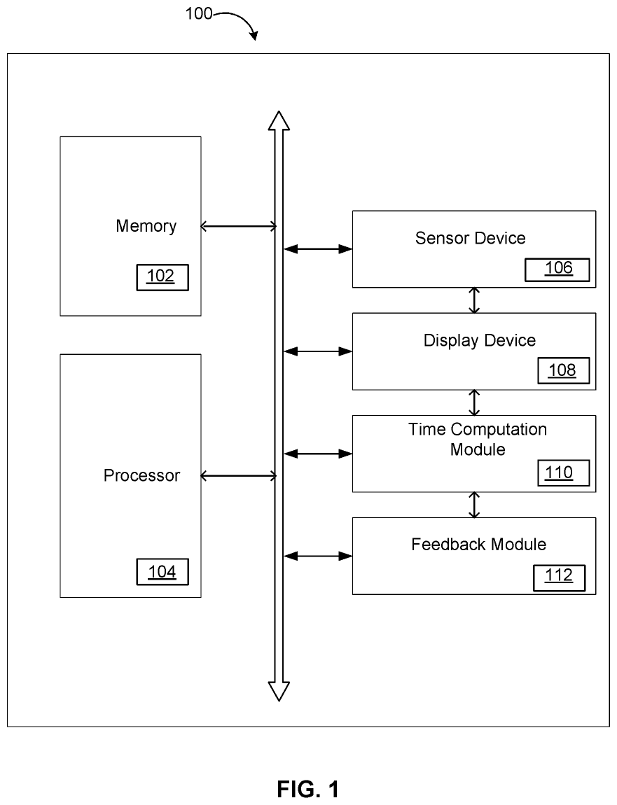

FIG. 1 illustrates a system 100 for navigating a user based on a type of maneuver for correction of a vestibular condition in accordance with an embodiment of the invention.

As illustrated in FIG. 1, system 100 includes a memory 102 and a processor 104 communicatively coupled to memory 102. System 100 further includes a sensor device 106 communicatively coupled to memory 102 and processor 104, sensor device 106 configured to collect sensor data regarding a head orientation and a body orientation of a person experiencing a vestibular condition, to be communicated to the user navigating the system for correction of vestibular condition. The user may be selected from a group of, but not limited to, a doctor, a physician, a clinician and an assistant.

In some embodiments, sensor device 106 includes at least two cameras for providing sensor data regarding a head orientation and a body orientation of the person. System 100 may further include a plurality of devices for determining the position, orientation and measurements of the person's head, body and eyes.

In a preferred embodiment, the sensor device comprises an augmented reality head gear device with a camera placed on a user's head for detecting the head orientation and body orientation of the person experiencing vestibular condition.

In accordance with system 100, processor 104 is further configured to create a three-dimensional model of the person experiencing vestibular condition, based on the collected sensor data pertaining to head orientation, body orientation and eye movements of the person. Further, a sequence of steps is generated by processor 104 in accordance with a type of maneuver to enable the user to perform each step of the sequence of steps. The type of maneuver is selected from a group of, but not limited to, Dix-Hallpike maneuver, an Epley maneuver, Canalith Repositioning, Semant maneuver, Barbecue maneuver, Gufoni maneuver or modifications thereof.

Accordingly, a display device 108 communicatively coupled to the memory 102, processor 104 and sensor device 106 is configured to display an animation corresponding to each step to be performed by the user, the animation overlaid on the three-dimensional model created of the person experiencing the vestibular condition. Sensor device 106 is further communicatively associated with a time computation module 110.

Time computation module 110 is configured to compute time duration of each step performed by the user, at the end of the performance of each step of the given sequence of steps. Time computation module 110 is further collaboratively coupled to a feedback module 112, configured to provide a real-time feedback based on a deviation between a set of predetermined sequence of steps and a set of actual sequence of steps as performed by the user. Feedback module 112 further provides a real-time feedback to the user on change in real time eye nystagmus and torsional eye movements during the performance of the actual sequence of steps by the user.

The real-time feedback from feedback module 112 further enables computation of an accuracy level of the performance of the sequence of steps based on a deviation between a set of predetermined steps and a set of actual steps corresponding to the type of maneuver, eye nystagmus and torsional eye movements of the person. Accordingly, based on the accuracy level of the performance of the sequence of steps, time duration of each step is adjusted, in collaboration with time computation module 110, thereby ensuring reduction in eye nystagmus of the person followed by complete zero nystagmus, confirming the completion of the performed maneuver.

In some embodiments, in accordance with system 100, sensor device 106 is an augmented reality head gear device with a camera placed on a user's head, employed for collecting sensor data regarding the head orientation and body orientation of the person experiencing a vestibular condition. The vestibular condition experienced by the person may be Benign Paroxysmal Positional Vertigo (BPPV) or a similar vestibular disorder. The augmented reality head gear device recognizes a head orientation and a body orientation of the person based on a marker position. The markers may be used in conjunction with a camera or the augmented reality head gear device.

In another embodiment, the augmented reality head gear device identifies the head and body orientations of the person without the use of markers.

In an example, the marker position includes a position on the head or torso of the person. A separate sensor device a plurality of cameras is also employed for collecting sensor data regarding eye movements to identify the presence of eye nystagmus and torsion in real-time.

On choosing a type of maneuver to be employed, the method navigates the user through each step of the sequence of steps, in accordance with the associated instruction set and time duration computed by time computation module 110, for performing the step. The augmented reality head gear device further enables a user to visualize the movement of the person, the movement relative to the sequence of steps generated by the processor-implemented method.

In an implementation, the sensor device is mounted on a person's head and the sensor device has infrared cameras which track the eye movements of the person to view nystagmus and torsion at each step of the maneuver. As different steps of the maneuver are completed, there may be changes in the eye nystagmus which indicate a completion of that step. The changes in eye nystagmus can be, but need not be limited to, change in number of beats per minute, change of Slow Phase Velocity (SPV) of nystagmus, change of intensity of nystagmus, change of direction of nystagmus, change of direction of torsion, change of frequency of torsion, change of intensity of torsion and a combination of two or more of the aforementioned changes.

Consider an example of a person experiencing symptoms of BPPV, wherein the person is seated on a bed. A physician addressing the person wears an augmented reality head gear device with plurality of embedded cameras that collects sensor data regarding the different orientations and eye movements associated with the person. Augmented reality head gear device recognizes the orientations and movements in a three-dimensional space and constructs a three-dimensional model of the person. A type of maneuver is then selected and a sequence of steps for performing the selected type of maneuver on the person is generated. Further, a time duration for performing each step of the sequence of steps is computed using time computation module 110 and provided for display to the physician on display device 108. Further, in accordance with the method and system an animation associated with each step is projected on the augmented reality head gear device. While the augmented reality head gear device is visualizing the movements and orientations of the person, feedback module 112 compares the pre-determined sequence of steps associated with the type of maneuver with the sequence of steps performed by the physician, further observing the time duration for the performance of each step. Based on the comparison, feedback module 112 provides a real-time feedback to the physician regarding the level of accuracy of the performance of steps with respect to movement, orientation as well as time duration of performance of each step computed by time computation module 110. Accordingly, time computation module 110 either adjusts the time duration of performance of the steps or confirms the correctness of the performance of steps of the type of maneuver, in response to the real-time feedback.

In some embodiments, in accordance with system 100, sensor device 106 is a pair of specially designed gloves, employed for providing sensor data regarding an orientation of the person's head and body, the person experiencing a vestibular condition. The specially designed gloves worn by the user may be associated with an augmented reality device, thereby enabling the user to visualize the movements made by the user with respect to the orientation of the person experiencing the vestibular condition.

Consider an example of a person experiencing symptoms associated with a vestibular disorder, seated on a bed. A clinician addressing the person is wearing a pair of specially designed gloves, further associated with an augmented reality device. The specially designed pair of gloves collects sensor data regarding the different orientations associated with the person based on the relative position of the clinician's hands on the person during the performance of steps in accordance with the pre-determined type of maneuver. Accordingly, an animation associated with each step is projected on a display device selected from an augmented device and a display screen. While the augmented device is visualizing the movements and orientations of the person, feedback module 112 compares the pre-determined sequence of steps associated with the type of maneuver with the sequence of steps performed by the clinician, further observing the time duration for the performance of each step. Based on the comparison, feedback module 112 provides a real-time feedback to the clinician regarding the level of accuracy of the performance of steps with respect to movement, orientation as well as time duration of performance of each step computed by time computation module 110.

Accordingly, time computation module 110 either adjusts the time duration of performance of the steps, or confirms the correctness of the performance of steps of the type of maneuver, in response to the real-time feedback received from feedback module 112.

In some embodiments, system 100 automatically provides an instruction set on selecting a type of maneuver, further computing time duration at the end of performance of each step, enabling system 100 to further instruct corrective measures to the user at real-time.

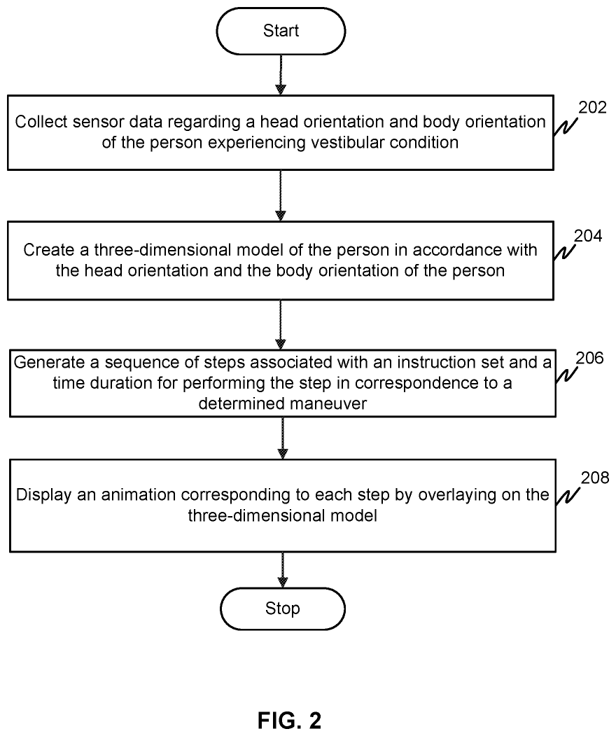

FIG. 2 illustrates a flow chart depicting a method for navigating a user based on a type of maneuver for correction of a vestibular condition, in accordance with system 100.

At an initial step, 202, processor implemented method collects sensor data regarding a head orientation and body orientation of the person experiencing vestibular condition. The sensor data collected at step 202 enables the processor in deriving the vestibular condition of the person experiencing the vestibular condition. The sensor data is collected by sensor device 106 that may include, but is not limited to, a plurality of cameras, a plurality of infrared cameras, an augmented reality head gear device with a camera and a pair of specially designed gloves. On receiving sensor data at step 202, the method creates a three-dimensional model of the person in accordance with the head orientation, the body orientation of the person and eye movements of the person experiencing the vestibular condition, at step 204. In an ensuing step, at step 206, a sequence of steps is generated corresponding to a type of maneuver. The type of maneuver may be pre-determined by the user. The pre-determination of the type of maneuver is based on the type of vestibular condition derived from a diagnosis conducted by the user. The pre-determined type of maneuver selected by the user may include, but is not limited to, Dix-Hallpike maneuver, an Epley maneuver, Canalith Repositioning, Barbecue maneuver, Gufoni maneuver, a Semant maneuver or modifications thereof

Each step of the sequence of steps generated at step 206, is further associated with an instruction set and a time duration for performing the step. The time duration for performing each step is computed by time computation module 110. Time computation module 110 is collaboratively coupled to a feedback module 112. In a concluding step, at step 208, the processor implemented method displays an animation corresponding to each step to be performed by the user for further overlaying of the animation on the three-dimensional model of the person experiencing a vestibular condition, generated at step 204.

Once the user performs the sequence of steps generated in accordance with the instruction set and time duration for the performance of each step, feedback module 112 provides a real-time feedback based on a deviation between a set of predetermined sequence of steps and a set of actual sequence of steps as performed by the user. Feedback module 112 also provides a real-time feedback to the user on change in real time eye nystagmus and torsional eye movements during the performance of the actual sequence of steps by the user, thereby enabling computation of an accuracy level in accordance with the deviation. Furthermore, time computation module 110 in collaboration with feedback module 112, adjusts time duration for performance of each step by the user, ensuring reduction in eye nystagmus and torsional eye movements of the person followed by complete zero nystagmus. The person experiencing zero nystagmus is confirmative of the completion of the type of maneuver performed by the user.

The present invention advantageously provides an appropriate corrective mechanism for adjusting the sequence of steps in terms of person orientation as well as time duration spent in the performance of the steps, thereby maintaining a relatively high level of accuracy.

The present invention further provides a cost-effective and economical methodology as the user is provided with an on-going, real-time feedback as and when each step is performed in accordance with a type of maneuver, thereby mitigating the need for extensive training of users for performance of the steps.

Those skilled in the art will realize that the above recognized advantages and other advantages described herein are merely exemplary and are not meant to be a complete rendering of all of the advantages of the various embodiments of the invention.

The system, as described in the invention or any of its components may be embodied in the form of a computing device. The computing device can be, for example, but not limited to, a general-purpose computer, a programmed microprocessor, a micro-controller, a peripheral integrated circuit element, and other devices or arrangements of devices, which are capable of implementing the steps that constitute the method of the invention. The computing device includes a processor, a memory, a nonvolatile data storage, a display, and a user interface.

In the foregoing specification, specific embodiments of the invention have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of the invention. The benefits, advantages, solutions to problems, and any element(s) that may cause any benefit, advantage, or solution to occur or become more pronounced are not to be construed as a critical, required, or essential features or elements of any or all the claims. The invention is defined solely by the appended claims including any amendments made during the pendency of this application and all equivalents of those claims as issued.

We claim:

1. A method for navigating a user based on a type of maneuver for correction of a vestibular condition, the method comprising:
collecting, by one or more processors, sensor data regarding one of a head orientation and a body orientation of a person;
creating, by one or more processors, a three-dimensional model of the person in accordance with the head orientation and the body orientation of the person;
generating, by one or more processors, a sequence of steps corresponding to the type of maneuver, wherein each step of the sequence of steps is associated with an instruction set and a time duration for performing the step; and
enabling, by one or more processors, the user to perform each step of the sequence of steps, wherein the enabling comprises displaying sequentially, by one or more processors, an animation corresponding to each step to be performed by the user, the animation being overlaid on the three-dimensional model of the person.

2. The method according to claim 1, wherein a type of maneuver is one of a Dix-Hallpike maneuver, an Epley maneuver, Canalith Repositioning, a Semant maneuver. Barbecue maneuver and Gufoni maneuver.

3. The method according to claim 1, wherein a vestibular condition is Benign Paroxysmal Positional Vertigo (BPPV).

4. The method according to claim 1, wherein a sensor device is used for collecting the sensor data regarding one of the head orientation and the body orientation of the person.

5. The method according to claim 4, wherein the sensor device comprises at least two cameras for providing sensor data regarding an orientation of the person's head and body.

6. The method according to claim 4, wherein the sensor device comprises a pair of special designed gloves for providing sensor data regarding an orientation of the person's head and body.

7. The method according to claim 4, wherein the sensor device comprises a head gear with infrared cameras.

8. The method according to claim 4, wherein the sensor device is an augmented reality head gear device with a camera that is placed on the user's head for detecting the head orientation and the body orientation of the person.

9. The method according to claim 1, wherein the creating comprises generating the three-dimensional model of the person using an augmented reality head gear device.

10. The method according to claim 9, wherein the augmented reality head gear device recognizes a head orientation and a body orientation of the person based on at least one marker position.

11. The method according to claim 1, wherein a time duration for a step is computed based on the eye nystagmus and torsional eye movements of the person determined by eye tracking.

12. The method according to claim 1 further comprises providing, by one or more processors, a real-time feedback on an accuracy level with which a step is being performed, wherein the accuracy level is determined based on at least one of a deviation between a set of predetermined steps and a set of actual steps corresponding to the type of maneuver, eye nystagmus and torsional eye movements of the person.

13. The method according to claim 12 comprises providing further instructions for performing the step and adjusting a time duration for the step based on the accuracy level.

14. A system for navigating a user based on a type of maneuver for correction of a vestibular condition, the system comprising:
a memory;
a processor communicatively coupled to the memory;
a sensor device communicatively coupled to the memory and the processor, wherein the sensor device is configured to collect sensor data regarding one of a head orientation and a body orientation of a person;
wherein the processor is configured to:
create a three-dimensional model of the person in accordance with the head orientation and the body orientation of the person;
generate a sequence of steps corresponding to the type of maneuver, wherein each step of the sequence of steps is associated with an instruction set and a time duration for performing the step; and
enable the user to perform each step of the sequence of steps; and
a display device communicatively coupled to the memory, the processor and the sensor device, wherein the display device is configured to display sequentially an animation corresponding to each step to be performed by the user, the animation being overlaid on the three-dimensional model of the person.

15. The system according to claim 14, wherein the sensor device comprises least two cameras for providing sensor data regarding an orientation of the person's head and body.

16. The system according to claim 14, wherein the sensor device comprises a pair of special designed gloves for providing sensor data regarding an orientation of the person's head and body.

17. The system according to claim 14, wherein the sensor device comprises a head gear with infrared cameras.

18. The system according to claim 14, wherein the sensor device is an augmented reality head gear device with a camera that is placed on the user's head for detecting the head orientation and the body orientation of the person.

19. The system according to claim 14, wherein the processor is configured to generate the three-dimensional model of the person using an augmented reality head gear device.

20. The system according to claim 19, wherein the augmented reality head gear device recognizes a head orientation and a body orientation of the person based on at least one marker position.

21. The system according to claim 14, wherein a time duration for a step is computed based on the eye nystagmus and torsional eye movements of the person determined by eye tracking.

22. The system according to claim 14, wherein the processor is further configured to provide a real-time feedback on an accuracy level with which a step is being performed, wherein the accuracy level is determined based on at least one of a deviation between a set of predetermined steps and a set of actual steps corresponding to the type of maneuver, eye nystagmus and torsional eye movements of the person.

23. The system according to claim 22, wherein the processor is configured to provide further instructions for performing the step and to adjust a time duration for the step based on the accuracy level.
